(19) United States
(12) Patent Application Publication
(10) Pub. No.: US 2015/0213208 A1
(43) Pub. Date: Jul. 30, 2015

(54) WEARABLE DEVICE FOR MEDICAL CARE

(71) Applicant: HAN-WEI ZHANG, New Taipei City (TW)

(72) Inventor: HAN-WEI ZHANG, New Taipei City (TW)

(21) Appl. No.: 14/607,148

(22) Filed: Jan. 28, 2015

(30) Foreign Application Priority Data
Jan. 29, 2014 (TW) 103202095

(51) Int. Cl. G06F 19/00; A61B 5/022; A61B 5/00

(52) U.S. Cl. CPC G06F 19/34; A61B 5/0022; A61B 5/022

(57) ABSTRACT

The instant disclosure relates to a wearable device for medical care. The device can be worn by the medical staff to help care recipients. The device includes a measuring module, a storing module, and a processing module. The measuring module is utilized to examine the patient in obtaining character-based information. The storing module is used to store character-based data corresponding to the obtained character-based information. The processing module is in electrical connection with the measuring and storing modules. The processing module receives the character-based information from the measuring module, and converts the character-based information into character-based data for storing in the storing module. The conversion is performed after the medical staff has obtained the character-based information of the patient via the measuring module. Thus, the care of recipients by the medical staff can be facilitated, and erroneous data record due to human input can be avoided.

### CROSS-REFERENCES TO RELATED APPLICATIONS

This non-provisional application claims priority under 35 U.S.C. §119(a) on Patent Application No. 103202095 filed in Taiwan, R.O.C. on 2014 Jan. 29, the entire contents of which are hereby incorporated by reference.

### BACKGROUND

1. Technical Field

The instant disclosure relates to a wearable device, in particular, for medical care applications.

2. Related Art

Healthcare personnel, such as in hospitals, would often patrol patient rooms at regular intervals. During inspections, medical personnel normally roll a hospital cart to perform some basic check-ups on the patients. Some exemplary tasks are checking patients' blood pressure, taking temperature, changing wound dressing, etc. The resulting medical records are then stored in a computer on the hospital cart.

Besides storing medical devices, hospital carts normally have an extra space designated for mounting a computer. The computer allows medical personnel to record patients' health conditions, such as blood pressure, heartbeat, and temperature instantly. However, computer-equipped carts are often large in size and cumbersome. The other disadvantage is that healthcare personnel must key in patients' medical records into the computer manually, which could lead to human input error.

There are several known wearable measurement devices for checking wearers' health conditions, such as heartbeat, blood pressure, temperature, etc. In other words, after a user has put on a wearable measurement device, the device would self-measure the user's physiological condition and self-record the obtained data, such that the user can monitor his or her health condition in real time.

Nevertheless, aforementioned wearable measurement devices are operable only to the person wearing the devices. If it is intended for use by another person, the original user must remove the devices, so that the next person can wear these devices to take desired measurements. To change the user, the wearing and removing steps of such wearable measurement devices create an issue of inconvenience.

### SUMMARY

In view of the foregoing, the instant disclosure provides a wearable device for medical care (hereinafter referred to as “wearable device”). The wearable device is for wearing by a healthcare professional, such that various medical conditions of different individuals can be measured and recorded. By using the wearable device, healthcare personnel can take the measurements of different individuals more easily, while eliminating the risk of human input error. The facilitation of the medical measurement procedure is beneficial for the field of physical examination, health education, and health rehabilitation.

To achieve the above-mentioned attributes, the wearable device of the instant disclosure comprises a measuring module, a storing module, and a processing module. The measuring module takes measurements to obtain the physiological characteristics of tested individuals. The storing module records the physiological data corresponding to the measured characteristics, while the processing module is in an electrical connection with the measuring and storing modules. More specifically, the processing module receives the measured physiological characteristics from the measuring module, and converts the measured physiological characteristics to recordable data for saving in the storing module.

Based on the above configuration, the physiological information of tested individuals is obtained by the measuring module, and the physiological information is converted to physiological data by the processing module for storing in the storing module. The configuration provides a convenient approach for measuring the physiological characteristics of multiple individuals. The measured physiological characteristics of each tested individual are then self-recorded by the storing module. Therefore, healthcare professionals can care patients more easily and avoid human input error. Thus, healthcare related objectives such as physical examination, health education, and health rehabilitation.

### BRIEF DESCRIPTION OF THE DRAWINGS

FIG. 1 is a schematic view for a first embodiment of the instant disclosure.

FIG. 1A is a perspective view for the first embodiment of the instant disclosure.

FIG. 2 is a schematic view for a second embodiment of the instant disclosure.

FIG. 2A is another schematic view for the second embodiment of the instant disclosure.

FIG. 3 is a schematic view for a transmit module for the second embodiment of the instant disclosure.

FIG. 4 is another schematic view for the transmit module in FIG. 3.

FIG. 5 is yet another schematic view for the second embodiment of the instant disclosure.

FIG. 5A is still yet another schematic view for the second embodiment of the instant disclosure.

FIG. 6 is a schematic view for a third embodiment of the instant disclosure.

FIG. 7 is a schematic view for a fourth embodiment of the instant disclosure.

FIG. 8 is a schematic view for a fifth embodiment of the instant disclosure.

FIG. 9 is a schematic view for a sixth embodiment of the instant disclosure.

FIG. 10 is a schematic view for a seventh embodiment of the instant disclosure.

FIG. 11 is a schematic view for an eighth embodiment of the instant disclosure.

FIG. 12 is a schematic view for a ninth embodiment of the instant disclosure.

### DETAILED DESCRIPTION

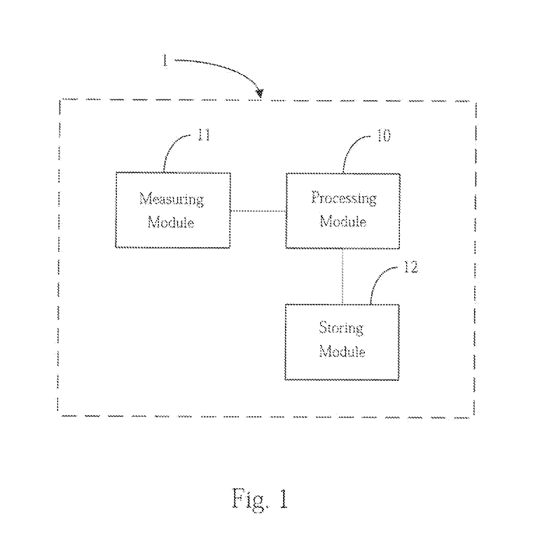

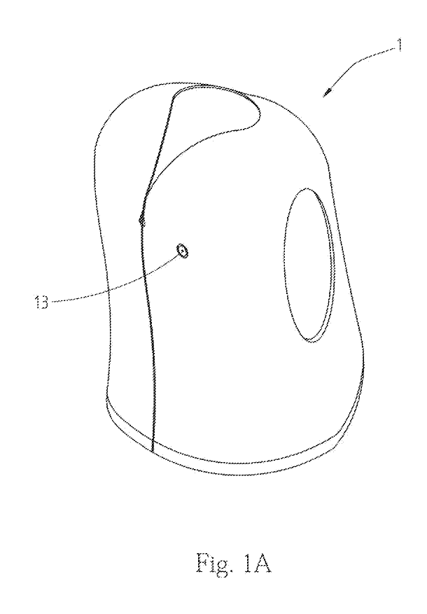

For the instant disclosure, FIG. 1 shows a schematic view of a first embodiment and FIG. 1A is a perspective view thereof. Please refer to FIG. 1, which shows a wearable device 1 comprising a measuring module 11, a storing module 12, and a processing module 10. The measuring module 11 measures an individual to obtain his or her physiological information. The storing module 12 records the physiological data corresponding to the obtained physiological information. The processing module 10 is connected electrically to the measuring module 11 and the storing module 12. More specifically, the processing module 10 receives the one or more recipients' physiological information from the measuring module 11, and converts the physiological information into physiological data to be recorded in the storing module 12. The wearable device 1 could be shaped as, but not limited to, a vest (as shown in FIG. 1A), a jacket, a waist belt, or any other wearable configuration.

The above-mentioned identification information could be physiological, ambient, or image related. For the physiological information, the range includes, but not limited to, blood pressure, body temperature, electrical activity of the heart, blood sugar concentration, pulse, blood oxygen level, and other physiological characteristics. Ambient information may include location, temperature, humidity, smoke distribution, carbon monoxide level, carbon dioxide level, or any other ambient condition characteristic. The measuring module 11 could be, but not limited to, a blood pressure machine, a medical thermometer, an electrocardiograph (ECG) machine, a blood sugar meter, an ambient thermometer, a humidity sensor, a camera, or any other device capable of obtaining physiological, ambient, or image-related characteristic.

The processing module 10 has a built-in measuring procedure, in correspondence to the measuring module 11. In other words, the processing module 10 initiates the measuring module 11 in accordance to the measuring procedure, such that the measuring module 11 can operate accordingly to collect the character-based information. For example, when the measuring module 11 is a blood pressure machine, the measuring procedure of the processing module 11 would command the cuff to be inflated above the likely systolic pressure. The cuff is then allowed to deflate slowly by releasing air from the cuff at a moderate rate, until the cuff pressure falls below the patient's diastolic pressure. The pulse measurement is made while the cuff is between the systolic and diastolic pressures. Thus, the systolic and diastolic pressure readings can be taken, along with the heartbeat rhythm. The preceding example is for explanation purposes only and is intended to be non-limiting. Based on the above, the healthcare personnel can conduct health examinations and provide measurement results to health educators. The measurement results can further be referenced for healthcare service and rehabilitation.

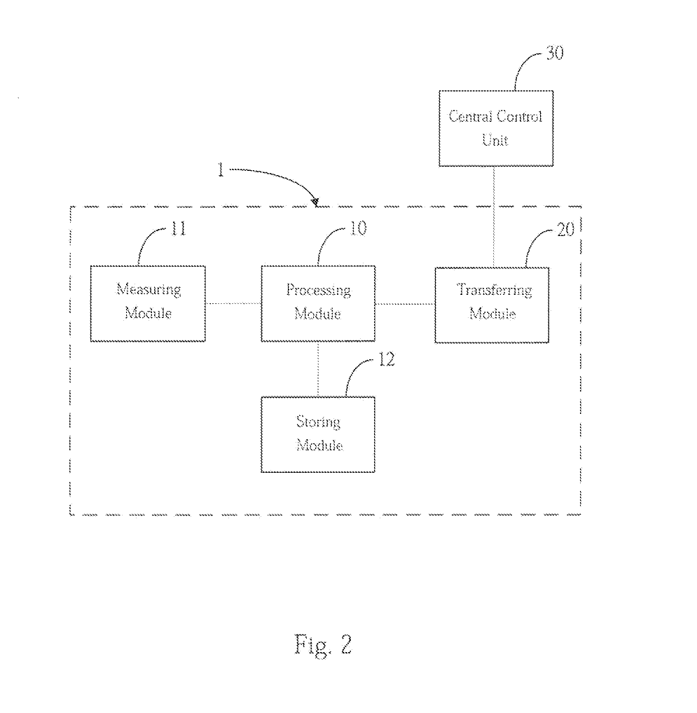

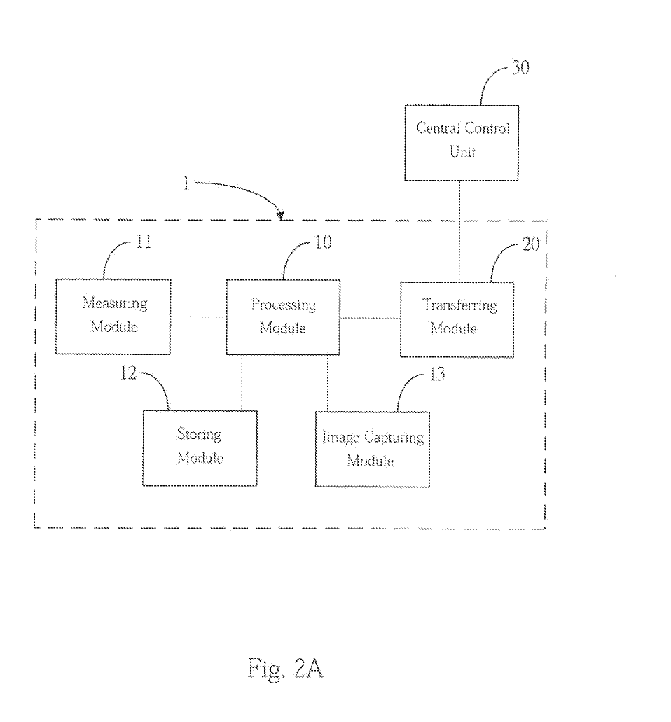

Please refer to FIG. 2, which shows a schematic view for a second embodiment of the instant disclosure. For this embodiment, the wearable device 1 further includes a transmitting module 20, which is connected electrically to the processing module 10. The transmitting module 20 is used to relay a character-based signal, which corresponds to the patient's physiological and ambient information, to a central control unit 30. The processing module 10 receives the patient's physiological and ambient information from the storing module 12, and converts the received information to the corresponding character-based signal. This signal is then relayed by the transmitting module 20 to the central control unit 30. Based on the received character-based signal, the central control unit 30 can be utilized to manage the measured data, such as creating extra copies, remote controlling, or performing analysis but not limited thereto. Also, the monitoring personnel may send a control signal to the transmitting module 20 via the central control unit 30, in order to control the operation of the wearable device 1. This is to say the monitoring personnel can remotely control the wearable device 1, in ways such as updating the parameter setting and changing the operation thereof, but are not limited thereto. The person wearing the wearable device 1 follows a specific walking route, but is not limited thereto. In some situations, he or she may take any route to anywhere without being constrained to a specific walking route. The wearable device 1, when signally connected with the central control unit 30, can synchronize its character-based signal to the central control unit 30. When the wearable device 1 is signally connected with the central control unit 30 in a continuous fashion, the character-based signal is synchronized to the central control unit 30 in real time. For example, but not limited thereto, a nurse in a hospital wears the wearable device 1. When the nurse is patrolling the patient rooms, the wearable device 1 can measure the blood pressure, body temperature, heart rate or other physiological information, along with the ambient condition information, of each patient. These measurements are then self-recorded into the storing module 12. As the nurse returns to the nursing station, the wearable device 1 will automatically be in communication with the central control unit 30. Then, the wearable device 1 will synchronize the stored information to the central control unit 30. In this manner, the doctors and nurses can easily retrieve the health history of one particular patient, such as his or her physical condition at earlier points in time, via the central control unit 30. For advantages, time spent on recording the medical data by nurses can be saved, and human input error can be avoided. The hospital records for patients can also be easily consolidated. In addition, for the medical staff, the instant disclosure offers a way to self-record the job assignments and work performed, which is convenient for future audit of work records and descriptions. The recorded work information may also serve as evidences to clarify any medical dispute.

Turning now to FIG. 2A, for the instant disclosure, this figure shows a schematic view for a variant of the second embodiment. The wearable device 1 in particular includes an image capturing module 13. The image capturing module 13 captures images as image information, which could be moving images, still images, or other image-related information. The range of images includes human portraits, bar codes, fingerprints, human iris images, or other image-related information. For the image capturing module 13, it may be a video camera, a still camera, a barcode reader, or any other image capturing device. With image capturing via the image capturing module 13, when any medical dispute occurs later, the captured images can tell the actions that took place to help clear up the facts. In addition, medical personnel at the central control unit 30 may view the captured images, to be in sync with the medical services provided. For example, medical personnel can be updated with patients' conditions in real-time, and offer needed assistance remotely.

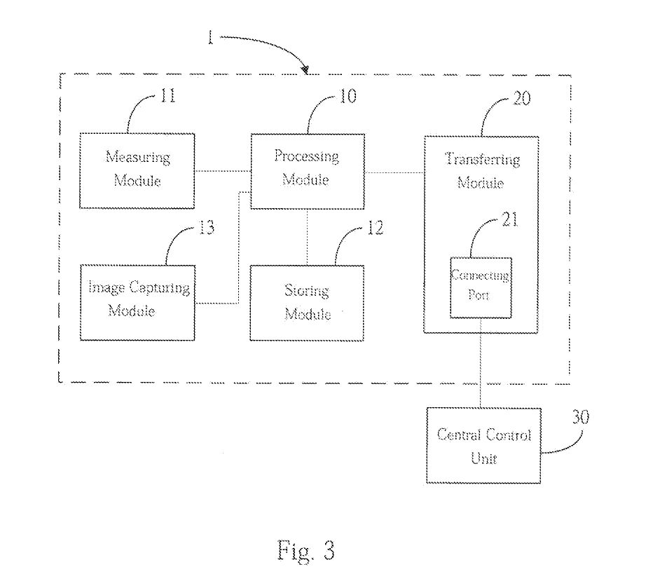

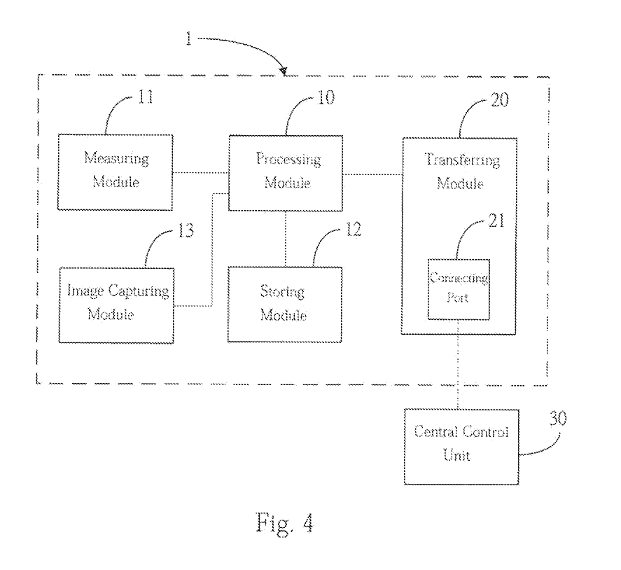

For the second embodiment of the instant disclosure, FIG. 3 shows a schematic view, in particular, for the transmitting module 20, and FIG. 4 illustrates a variant of the transmitting module 20. As shown in FIG. 3, the transmitting module 20 includes a connecting port 21, for plugging a transmission line 22 to transmit the character-based signal to the central control unit 30. In other words, wired connection is adopted between the transmitting module 20 and the central control unit 30, but is not limited thereto. Referring to FIG. 4, wireless signal transmit technology may be employed to transmit the character-based signal from the transmitting module 20 to the central control unit 30. For example, the wireless transmit technology may be Wi-Fi (wireless networking technology), Bluetooth, infrared, or other wireless transmit means without limitation.

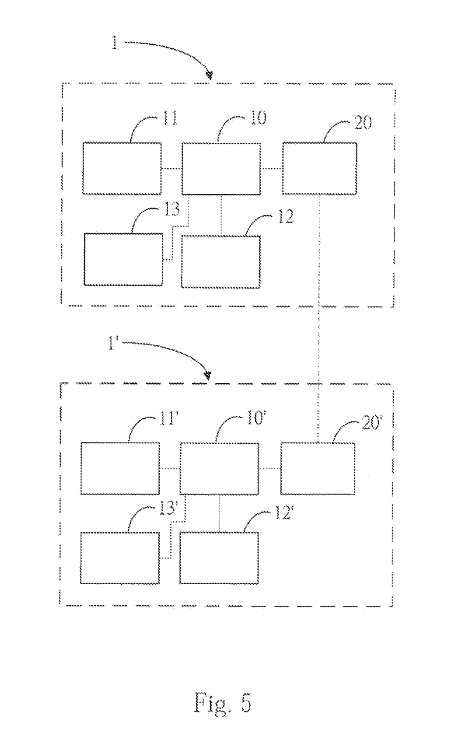

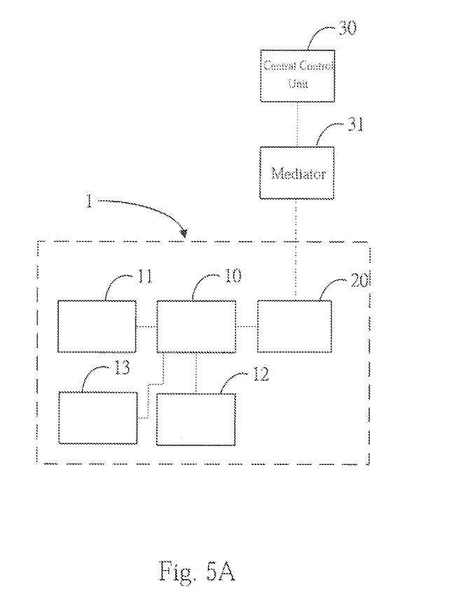

Referring to FIG. 5, another possible way of the second embodiment is illustrated. The transmitting module 20 of the wearable device 1 will, in a detection range, detect continuously if another wearable device 1′ is present. When the other wearable device 1′ is detected, the transmitting module 20 of the wearable device 1 will be signally connected with a transmitting module 20′ of the wearable device 1′. The signal connection enables the wearable device 1 to obtain the character-based information of the other wearable device 1′ and store in the storing module 12. Concurrently, the character-based information of the storing module 12 will be transmitted to the other wearable device 1′, via the transmitting module 20, and stored in a storing module 12′. In other words, if the wearable devices 1 and 1′ are worn by different individuals, and the wearable devices 1 and 1′ are in close proximity within a detectable range, the wearable devices 1 and 1′ will cross transmit its character-based information to one another. Thus, when one of the wearable devices 1 and 1′, such as the wearable device 1, is signally connected to the central control unit 30, the character-based information of the other wearable device 1′ will be transmitted to the central control unit 30 as well. This way, the central control unit 30 is able to receive various update character-based information, as quickly as possible. The preceding example is not meant to be in any way limiting. In circumstance where the internet service is unstable or inoperable at the hospital (e.g., distressful events or power outage), each nurse would usually be assigned to care a number of patients. When inspecting patient rooms, each nurse will check his or her patients and pass each other in the corridors. This fact means that for the wearable device 1 worn by a nurse, the corresponding character-based information can be transmitted to the wearable devices 1 worn by other nurses. Thereby, the nurse or nurses who return to the nursing station may transmit the character-based information of the patients and ambient conditions of the wearable devices 1 worn by the nurses who have not yet returned to the nursing station. In this manner, the central control unit 30 can be fed with the patient data as early as possible to perform the analysis. When any abnormality is found, a warning can then be issued as quickly as possible.

Referring to FIG. 5A, which is yet another variant of the second embodiment of the instant disclosure. In particular, a mediator 31 is signally connected between the transmitting module 20 and the central control unit 30. The mediator 31 receives the character-based signals, from the transmitting module 20, and transmits to the central control unit 30. Also, the mediator 31 receives control commands from the central control unit 30 and transmits to the transmitting module 20. That is, the mediator 31 is arranged at a fixed position, when the wearer passes by the mediator 31 within a detectable range, the wearable device 1, via the mediator 31, will automatically upload the character-based signals to the central control unit 30 or automatically receives the control commands therefrom. For example, in a hospital, at least one mediator 31 can be disposed in a corridor. Optionally each patient room or each hospital bed is furnished with one mediator 31. Still a further alternative is having one mediator 31 shared by a number of patient rooms or hospital beds. It is to be understood that the exact location and the number of mediators 31 are not in any way limited. Not shown is one or more wearable devices 1 may be designated to have the mediator 31, that is to say the mediator 31 is built-in within the wearable device 1. When one wearable device 1 without the mediator 31 and one wearable device 1 having the mediator 31 are in close proximity to each other and within a detectable range, the character-based information for the wearable device 1 without the mediator 31 will be transmitted to the wearable device 1 having the mediator 31 and stored in its storing module 12. By being integrated into the wearable device 1, the mediator 31 becomes mobile. So when the internet service at the hospital or medical treatment scenes is unstable or inoperable, the wearable device 1 with mediating capability may serve as a temporary server. After the internet connection is restored, the character-based information of the wearable device 1 with mediating capability can then be synchronized to the central control unit 30. For cases where the mediator 31 is not built within the wearable device 1, the wearable device 1 is usually connected to the mediator 31 in a wireless manner. For special or emergency circumstances, in contrast, a wired connection is preferred to maintain the stability of signal transmission between the wearable device 1 and the mediator 31, to avoid wireless connection that is more susceptible to instability in the signal transmission. An example would be the mediator 31 is arranged by a hospital bed. When the patient is experiencing an emergency situation, the nurse may use wired connection to signally connect the wearable device 1 to the mediator 31. Thus, the character-based signal can be transmitted in a stable manner to seek assistance from other medical members (e.g., nurses or physicians) at the central control unit 30 (e.g., nursing station). An example may be a remote assistance through voice communication.

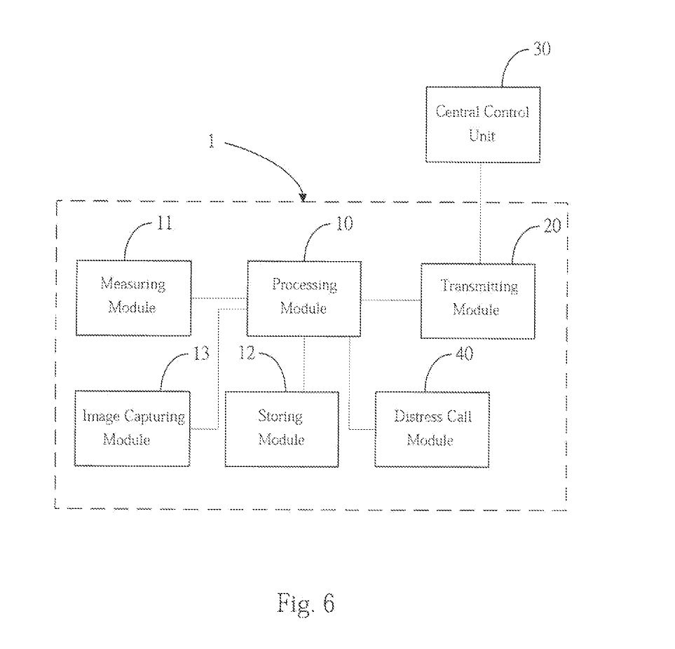

Referring to FIG. 6, a third embodiment of the instant disclosure is illustrated. In this embodiment, the wearable device 1 further includes a distress call module 40, which is connected electrically to the processing module 10. The distress call signal 40 generates a distress call signal in the event of an emergency. This signal is transmitted to the central control unit 30 via the transmitting module 20. For example, when the wearer is caring one or more patients, if the patient has an emergency condition requiring extra assistance or the wearer is under life threatening situation, the distress call module 40 can be utilized to generate the distress call signal. Thus, physicians, nurses, or security members at the central control unit 30 may arrive the scene as quickly as possible. The distress call module 40, not limited thereto, may be a button or any other means that can be touch-activated in an easy and convenient manner, in order to generate the distress call signal. Although each hospital bed has an assigned emergency call button, but this call button is unreachable from outside of patient rooms. That is, when the medical staff is outside of patient rooms, in the event of an emergency or additional assistance is needed, help request can be forwarded to the central control unit 30, via the distress call module 40 of the wearable device 1, to answer patient needs quickly.

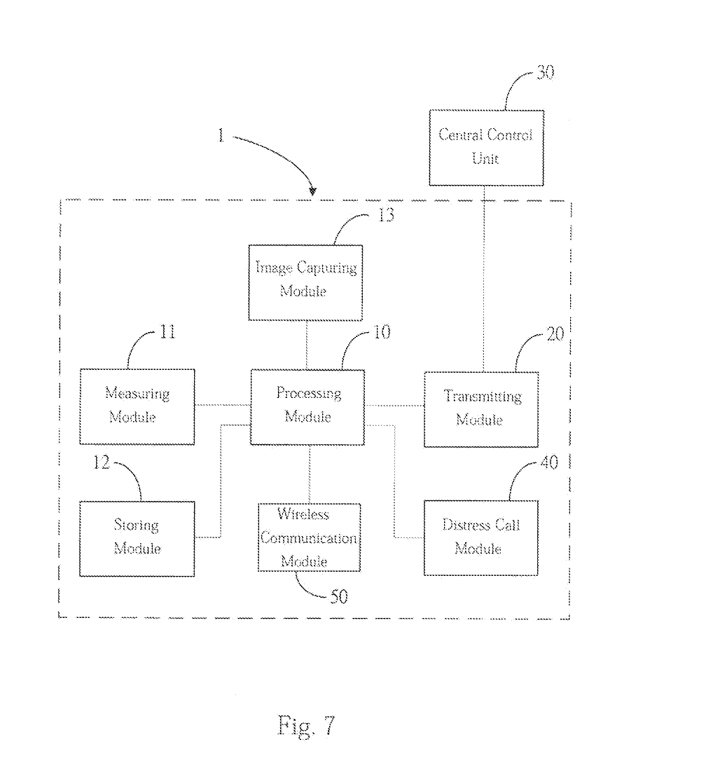

Referring to FIG. 7, a fourth embodiment of the instant disclosure is shown, in which the wearable device 1 further includes a wireless communication module 50. The wireless communication module 50 is in electrical connection with the processing module 10. Via the wireless communication module 50, the processing module 10 receives a voice signal and passes to the transmitting module 20, in order to forward the voice signal to the central control unit 30. In this way, voice communication is enabled between the staff at the central control unit 30 and the individual wearing the wearable device 1.

More specifically, the wireless communication module 50 receives audio waves nearby the wearable device 1, in which the audio waves are generated by the wearer or patients. The wireless communication module 50 then converts the audio waves into the voice signal for transmitting to the processing module 10, such that the voice signal can be transmitted to the central control unit 30 via the transmitting module 20. Based on the same principle, the central control unit 30 is capable of converting received audio waves into a voice signal. The voice signal is received by the transmitting module 20 and via the processing module 10, is transmitted to the wireless communication module 50. The wireless communication module 50 then converts received voice signal into audio waves and play the voice message. The voice message can then be heard by the wearer or individuals nearby the wearable device 1. This capability enables voice communication between the wearer or individuals near the wearable device 1 with the staff at the central control unit 30 or other wearers. Optionally the wireless communication module 50 can operate jointly with the image capturing module 13. That is to say, in addition to above-described two-way voice communication, the central control unit 30 can also receive images to get a better understanding of the scene. Since the wireless communication module 50 is a known technology to one skilled in the art, no further elaboration will be given herein.

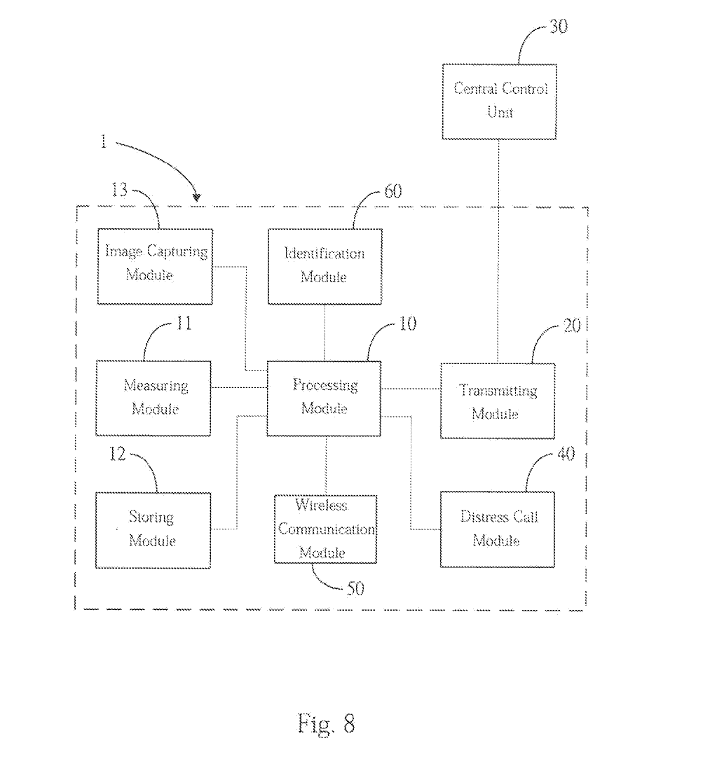

Referring to FIG. 8, a fifth embodiment of the instant disclosure is provided.

The wearable device 1 further includes an identification module 60, which is connected electrically to the processing module 10. In particular, a multiplicity of images is stored in the storing module 12. For the instant embodiment, the identification module 60 reads an image and cross correlates with stored images to identify the matching image. Then, an identification code is generated from the processing module 10, to quickly obtain the identification information corresponding to the image read by the identification module 60. Thus, the medical staff can perform real time-verification of the identification information. Also, the identification code can be transmitted to the central control unit 30 via the transmitting module 20. It is to be understood that the cross-correlation can also be done by the processing module 10, but is not limited thereto. In other words, the processing module 10 has the capability of the identification module 60. In some cases, the processing module 10 has the identification and cross-correlation capabilities. This configuration implies the identification module 60 may be built in within the processing module 10. In operation, after the image capturing module 13 has captured the image (e.g., portraits, barcodes, fingerprints, human iris images, or other types of image), the captured image is transmitted to the identification module 60 via the processing module 10 for cross-correlation. In addition, the captured image can be transmitted to the central control unit 30 via the processing module 10 and the transmitting module 20, for creating extra copies, monitoring, etc. The resulting benefit is the staff at the central control unit 30 can visually monitor the scene via the wearer, in addition to voice communication with the wearer via the wireless communication module 50. Such capabilities further facilitate the provision of technical assistance from the central control unit 30, so that the wearer can receive improved instructions any time to care patients. An example is provided herein, but is not limited thereto. When the medical staff first put on the wearable device 1, the image capturing module 13 can be utilized to capture an image (e.g., facial image or barcode) for identification purpose. That is to say the wearable device 1 first records which medical staff is wearing the wearable device 1. The identification information is then passed to the central control unit 30 via the transmitting module 20 for record keeping. Next, when the wearer is inspecting patients, each patient can be identified through the identification module 60, before taking measurements to obtain character-based information of patients. Thus, the identifications of the medical staff and patients, character-based information, time, and so forth, can be stored in the storing module 12. Such aspect can prevent the medical staff from making human-input error. The other advantage is a health history of each patient can be compiled for evaluation by physicians, in order to provide health educational services. Furthermore, based on the established health history, appropriate decisions regarding medical procedures, health care services, and health rehabilitation could be made accordingly.

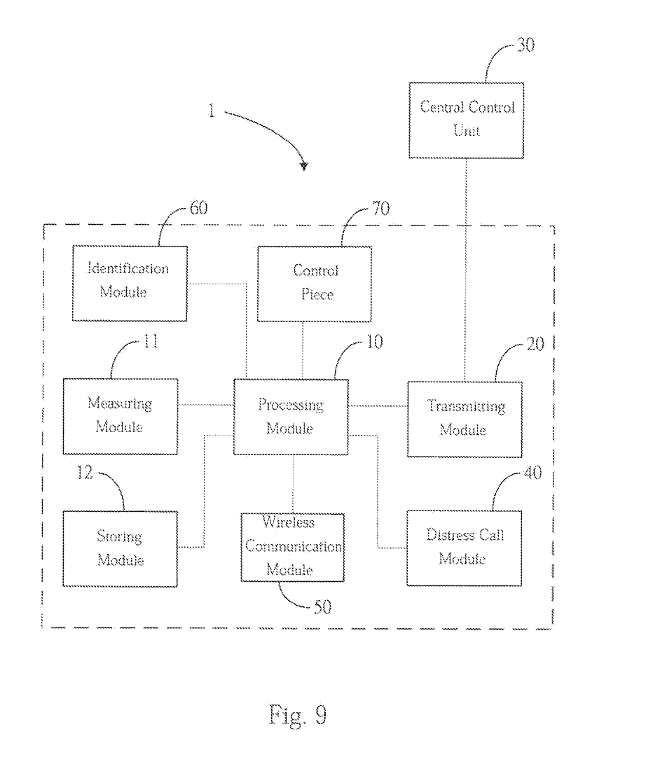

Referring to FIG. 9, a sixth embodiment of the instant disclosure is shown. The wearable device 1 further includes a control piece 70, which is in electrical connection with the processing module 10. The control piece 70 is employed to set and control various parameters or on/off arrangement of the preceding modules of the wearable device 1. The modules referred to herein include the processing module 10, the measuring module 11, the storing module 12, the transmitting module 20, the distress call module 40, the wireless communication module 50, and the identification module 60. The control pieces 70 may be, but not limited thereto, a button, a keyboard, a mouse (pointing device), a trackball, a touch screen, or other control device capable of making control arrangements.

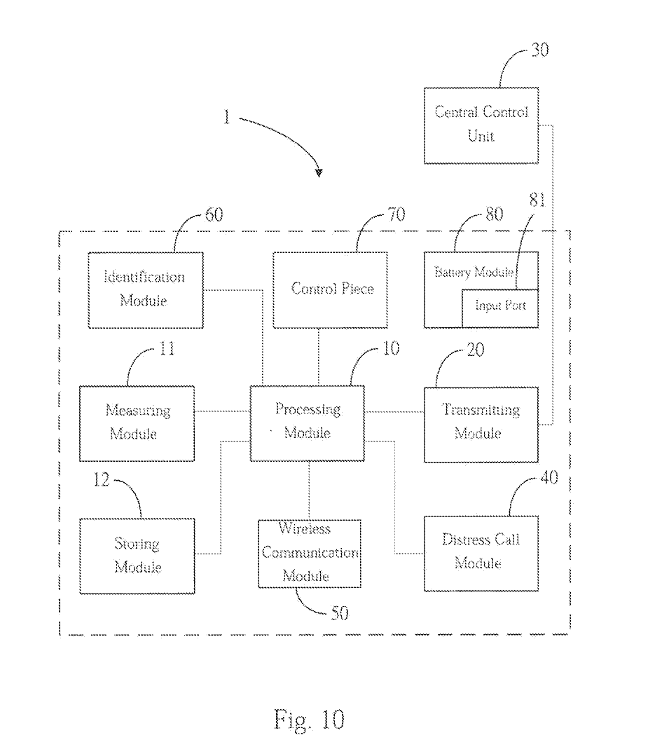

Turning now to FIG. 10, which illustrates a seventh embodiment of the instant disclosure. The wearable device 1 further includes a battery module 80. The battery module 80 has a built-in power source to power the wearable device 1. The battery module 80 is rechargeable by a chargeable power source. For normal operation, the battery module 80 is connected electrically to each of the modules of the wearable device 1, such as the processing module 10, the measuring module 11, the storing module 12, the transmitting module 20, the distress call module 40, the wireless communication module 50, and the identification module 60. The battery module 80 has an input port 81, for receiving a power cable to transmit power to the battery module 80. However, the instant disclosure is not limited to the above configuration. Not shown is the battery module 80 adopting wireless recharging technology. For example, upon returning to the nursing station, the nurse may take off the wearable device 1 and let it be hung by a cloth hanger.

The hanger is furnished with a wireless charging device for the battery module 80. So when the wearable device 1 is hung by the hanger, wireless recharging can be implemented. Alternatively, the wearable device 1 can be recharged by city power via the input port 81, as best illustrated in FIG. 10. Based on the above-described recharging means, the battery module 80 is kept from running out of power.

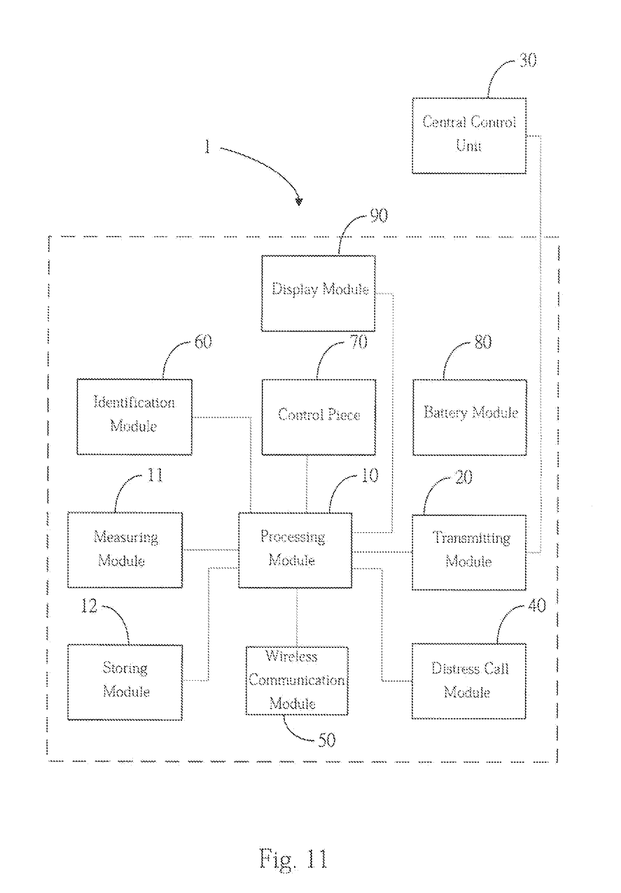

Referring to FIG. 11, an eight embodiment of the instant disclosure is shown. For this embodiment, the wearable device further includes a display module 90. Connected electrically to the processing module 10, the display module 90 displays the character-based information/data or the status of each module associated with the wearable device 1, including the processing module 10, the measuring module 11, the storing module 12, the transmitting module 20, the distress call module 40, the wireless communication module 50, the identification module 60, the control piece 70, and the battery module 80. That is to say, various information associated with the wearable device 1 can be displayed via the display module 90, for quick notification to the wearer or patients. Examples of the display module 90 are, but not limited thereto, liquid crystal display (LCD), touch screen, flexible display, and any other device capable of displaying content. Furthermore, the display module 90 may work in conjunction with the control piece 70 to control each of the modules associated with the wearable device 1 like the processing module 10, the measuring module 11, the storing module 12, the transmitting module 20, the distress call module 40, the wireless communication module 50, the identification module 60, and the battery module 80. In this manner, the wearer can easily control the wearable device 1. The display module 90 can be secured to the arm (e.g., upper arm or forearm) of the wearer, such that the displayed content by the display module 90 can be easily viewed by the wearer.

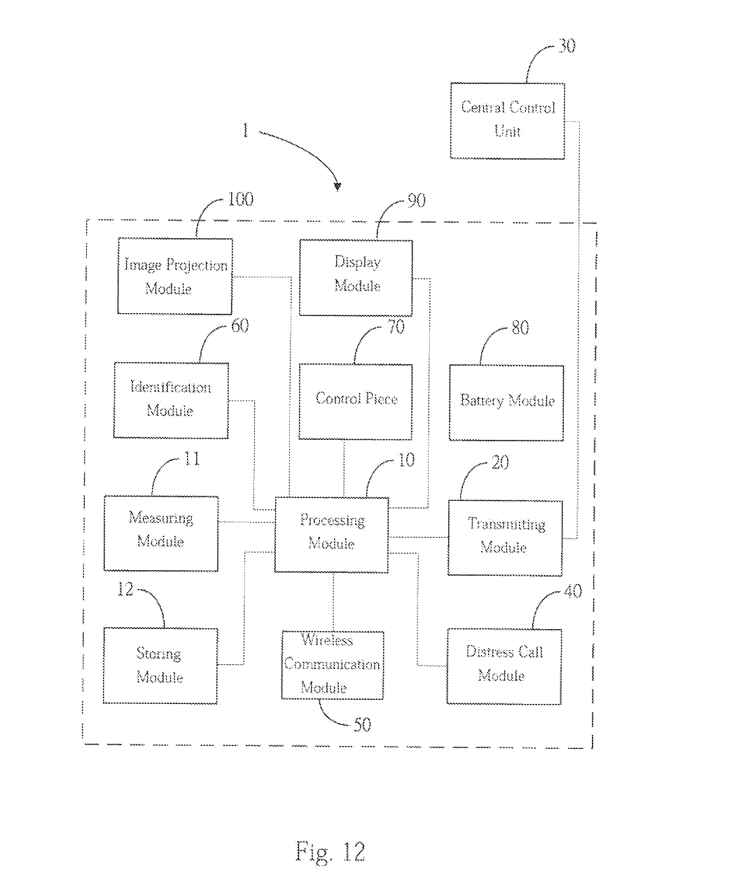

Referring to FIG. 12, a ninth embodiment of the instant disclosure is illustrated. For this embodiment, the wearable device 1 further includes an image projection module 100, which is in electrical connection with the processing module 10. The image projection module 100 is utilized for projecting images that include the information in association with the wearable device 1. More specifically, after the processing module 10 has retrieved various information of the patient via the storing module 12 or various signals, the retrieved content is converted by the processing module 10 into an image signal. The image projection module 100 is then utilized to project the images. That is, the image projection module 100 can project the information in association with the processing module 10, the measuring module 11, the storing module 12, the transmitting module 20, the distress call module 40, the wireless communication module 50, the identification module 60, the control piece 70, the battery module 80, or a combination thereof onto a projection screen, so a group of individuals may view the images together. The image projection module 100 may further operate with the wireless communication module 50 to carry out video chat or video conference, after the processing module 10 has passed the image signal to the central control unit 30 via the transmitting module 20.

It should be understood that the recipients mentioned hereinabove refer to patients, but is not limited thereto. For other circumstances, the recipients may also be infants, elders, or anyone who is checked for his/her physical conditions (e.g., someone taking a physical examination).

It should also be noted that the medical staff mentioned hereinabove refer to nurses, but is not limited thereto. In other situations, medical staff may be physicians, caregivers, family members, or anyone capable of wearing the wearable device 1. That is, anyone who wears the wearable device 1 may take above-described measurements from the recipients.

Based on the above, the wearable device 1 is worn by the wearer. Measurements associated with character-based information are taken by the measuring module 11, and the obtained character-based information is converted to character-based data by the processing module 10. The character-based data is then stored in the storing module 12. In addition to measuring the character-base information of the wearer, the wearable device 1 can also measure the character-based information of more than one patient. The measured patient information is also automatically stored in the storing module 12. For advantages, the care of patients by the wearer can be facilitated and human input-error can be avoided. Also, based on the obtained patient data, appropriate medical procedures, health education services, health care, and health rehabilitation can be provided. Furthermore, the obtained data can help clarify any medical dispute issue.

While the instant disclosure has been described by way of example and in terms of the preferred embodiments, it is to be understood that the invention needs not be limited to the disclosed embodiments. For anyone skilled in the art, various modifications and improvements within the spirit of the instant disclosure are covered under the scope of the instant disclosure. The covered scope of the instant disclosure is based on the appended claims.

What is claimed is:

1. A wearable device, suitable for wearing by a medical staff to care one or more recipients, comprising:
a measuring module to obtain a character-based information from the recipient;
a storing module to store a character-based data corresponding to the character-based information; and
a processing module connected electrically to the measuring module and the storing module, for receiving the character-based information and converting the character-based information to the character-based data, wherein the character-based data is stored in the storing module.

2. The wearable device of claim 1, further comprising a transmitting module connected electrically to the processing module, the transmitting module transmits a character-based signal corresponding to the character-based information to a central control unit or receives the character-based data from the storing module and converts the character-based data to the character-based signal and transmits the character-based signal to the central control unit.

3. The wearable device of claim 2, wherein the transmitting module detects for one other wearable device in a detectable range for exchanging respective character-based signals and storing in the respective storing modules.

4. The wearable device of claim 2, wherein the transmitting module has a connecting port, for plugging a transmission cable to transmit the character-based signal to the central control unit.

5. The wearable device of claim 2, wherein the transmitting module receives a control command from the central control unit for controlling or updating the wearable device remotely.

6. The wearable device of claim 2, further comprising a mediator in between the central control unit and the transmitting module, wherein the mediator receives the character-based signal from the transmitting module and synchronizes the character-based signal to the central control unit, and wherein the mediator receives a control command from the central control unit and transmits to the transmitting module.

7. The wearable device of claim 2, wherein a mediator is built therein to receive the character-based signal from the transmitting module of one other wearable device and synchronize the character-based signal of the other wearable device to the central control unit, and wherein the mediator receives a control command from the central control unit and transmits the control command to the transmitting module of the other wearable device.

8. The wearable device of claim 2, further comprising a distress call module in electrical connection with the processing module, wherein the distress call module generates a distress call signal and transmits to the central control unit via the transmitting module.

9. The wearable device of claim 1, further comprising an identification module in electrical connection with the processing module, wherein a plurality of image data is built within the storing module, wherein the identification module reads an image and cross-correlates with the storing module to find the matched image data, in order to generate an identification code via the processing module.

10. The wearable device of claim 1, further comprising an image capturing module connected electrically to the processing module, wherein the image capturing module captures an image as the character-based information and transmits the character-based information to the processing module.

11. The wearable device of claim 10, further comprising a control piece connected electrically to the processing module, wherein the control piece sets the parameters of the wearable device or on/off arrangements of the image capturing module.

12. The wearable device of claim 1, wherein the processing module has a built-in measuring procedure for initiating the measuring module to obtain the character-based information.

13. The wearable device of claim 1, further comprising a battery module having a power source for operating the wearable device, wherein the battery module is recharged electrically by an electrically chargeable device.

14. The wearable device of claim 13, wherein the battery module has an input port for receiving a power cable to electrically recharge the battery module.

15. The wearable device of claim 13, wherein the battery module is electrically recharged wirelessly.

16. The wearable device of claim 1, further comprising a display module connected electrically to the processing module for displaying the contents of the wearable device.

17. The wearable device of claim 1, further comprising an image projection module connected electrically to the processing module for projecting the contents of the wearable device.

18. The wearable device of claim 2, further comprising a wireless communication module connected electrically to the processing module, wherein the wireless communication module converts received sound waves to a voice signal, wherein the voice signal is transmitted to the transmitting module via the processing module, and wherein the wireless communication module is also capable of receiving a voice signal from the transmitting module and converts the voice signal to a sound wave for playing the sound wave.

19. The wearable device of claim 1, wherein the character-based information includes physiological information or ambient condition information.
